(19) United States
(12) Patent Application Publication
(10) Pub. No.: US 2007/0269496 A1
(43) Pub. Date: Nov. 22, 2007

(54) Patch for Reducing Exposure of Skin to Ultraviolet Radiation

(75) Inventor: Reed Gamble, Bilsdale (GB)

Correspondence Address: PATENT ADMINISTRATOR;KATTEN MUCHIN ROSENMAN LLP, 1025 THOMAS JEFFERSON STREET, N.W., EAST LOBBY: SUITE 700, WASHINGTON, DC, 20007-5201, US

(73) Assignee: Gamble De Grussa Ltd.

(21) Appl. No.: 10/573,247

(22) PCT Filed: Sep. 22, 2004
(86) PCT No.: PCT/GB04/04034

§ 371 Date: Apr. 2, 2007

(30) Foreign Application Priority Data
Sep. 23, 2003 (GB) 03223427

(51) Int. Cl. A61K 9/70; A61P 17/16; A61P 35/00; A61Q 17/00

(52) U.S. Cl. 424/443; 424/59

(57) ABSTRACT

A patch for reducing exposure to ultraviolet (UV) radiation includes a first layer that is adhesive. The patch includes a second layer. The second layer includes a material adjacent to the first layer. At least one of the first and second layers is opaque to UV radiation.

The invention relates to a patch, kit or method for reducing exposure of skin to UV radiation.

### BACKGROUND TO THE INVENTION

Protecting individuals from sunlight is important due to the deleterious cosmetic and medical effects of sunlight on skin and subcutaneous tissues, both immediately after exposure and after prolonged and/or repeated exposure. Cosmetically, sunlight can cause reddening of the skin (erythema) and repeated exposure can cause premature aging.

Sun light is composed of a continuous spectrum of electromagnetic radiation composed of 66% infra-red light (manifested as heat), 32% visible light and 2% ultraviolet light (UV). The UV spectrum consists of; UVA1 (340-400nm), and UVA2 (320-340 nm), UVB (280-320 nm) and UVC (200-280 nm). UV light has been shown to cause deleterious medical effects, both UVA and UVB have been found to cause long term damage to skin cells by inducing DNA lesions such as pyrimidine dimers and photoproducts which could lead to DNA mutations and skin cancer if not repaired. UVA has a longer wavelength than UVB and can penetrate deeper into the skin. Whilst UVB has been shown to be the main cause of erythema, the action spectrum of erythema is between 290-330 nm which also includes the shorter UVA wavelengths, ie UVA2. The UVC component of sunlight also causes deleterious medical effects, but this element is effectively filtered by the stratospheric ozone layer.

There are three principal types of skin cancer, basal cell and squamous cell carcinomas and melanoma. Basal cell and squamous carcinomas are generally non-aggressive and thus are seldom fatal although they can be disfiguring. Melanoma affects the melanocyte cells which produce melanin and can spread to affect the liver, lungs or brain. Melanoma begins when melanocytes gradually become mutated and unstable and divide without control or order. These cells can invade and destroy the normal cells around them. The abnormal cells form a growth of malignant tissue (a cancerous tumour) on the surface of the skin. These are called ‘melanomas’. Melanomas can appear suddenly with no warning or can develop from or around moles.

Melanomas fall into four basic categories as outlined below;

“Superficial spreading melanoma” is by far the most common type, accounting for about 70 percent of all cases. This melanoma travels along the top layer of the skin for a fairly long time before penetrating more deeply. The first sign is the appearance of a flat or slightly raised discoloured patch that has irregular borders and is somewhat geometrical in form. The colour varies, and you may see areas of tan, brown, black, red, blue, or white. Sometimes an older mole will change in these ways, or a new one will arise. The melanoma can be seen almost anywhere on the body, but is most likely to occur on the trunk in men, the legs in women, and the upper back in both. Most melanomas found in the young are of the superficial spreading type.

“Lentigo maligna” accounts for about 10% of melanoma in the UK and is similar to the superficial spreading type, as it also remains close to the skin surface for quite a while, and usually appears as a flat or mildly elevated mottled tan, brown, or dark brown discoloration. This type of in situ melanoma is found most often in the elderly, arising on chronically sun-exposed, damaged skin on the face, ears, arms, and upper trunk. Lentigo maligna melanoma is the invasive form.

The third type of melanoma, “acral lentiginous melanoma”, also spreads superficially before penetrating more deeply. It is quite different from the others, though, as it usually appears as a black or brown discoloration under the nails or on the soles of the feet or palms of the hands. This type of melanoma is sometimes found in dark-skinned people. It is the most common melanoma in African-Americans and Asians, and the least common among Caucasians.

Unlike the other three types, “nodular melanoma”, is usually invasive at the time it is first diagnosed. This accounts for about 1 in 4 melanomas (25%) in the UK. The malignancy is recognized when it becomes a bump. The colour is most often black, but occasionally is blue, grey, white, brown, tan, red, or skin tone. The most frequent locations are the trunk, legs, and arms, mainly of elderly people, as well as the scalp in men. This is the most aggressive of the melanomas, and is found in 10 to 15 percent of cases.

The medical consensus is that very strong evidence supports the assertion skin cancers are caused by damage from UV rays in sunlight. In addition studies have shown that there are a number of genetic and individual risk factors that have been shown to correlate with the likelihood of developing malignant melanoma Risk factors include;
fair skin
blue, green or hazel eyes
light-coloured or red hair
tendency to burn rather than suntan
history of severe sunburns
many moles
freckles
a family history of skin cancer
high, intermittent exposure to solar UV

Moles are growths on the skin and are also known as nevi or nevus—singular. These growths occur when cells in the skin, called melanocytes, grow in a cluster with tissue surrounding them. Moles are usually pink, tan, brown, or flesh-coloured. Melanocytes are also spread evenly throughout the skin and produce the pigment, melanin, which gives skin its natural colour. When skin is exposed to the sun, melanocytes produce more melanin, causing the skin to tan, or darken.

Moles are very common. Most people have between 10 and 40 moles. A person may develop new moles from time to time, usually until about age 40. Moles can be flat or raised. They are usually round or oval. Many moles begin as a small, flat spot and slowly become larger in diameter and raised. Over many years, they may flatten again, become flesh-coloured, and disappear.

About one out of every ten people has at least one unusual (or atypical) mole that looks different from an ordinary mole. The medical term for these unusual moles is dysplastic nevi. Doctors believe that dysplastic nevi are more likely than ordinary moles to develop into melanoma

The skin has a number of inherent defence mechanisms to combat the effect of UV radiation, these mechanisms are outlined below;

Tanning

The skin uses a pigmentation system to darken the skin and reduce the transmission of UV light so protecting the nuclei from DNA damage. This involves specialised cells in the epidermis called melanocytes, which produce a UV absorbing polymer called melanin. Within seconds of UV irradiation immediate oxidisation of the melanin granules near the skin surface takes place which produces a tan that will develop in an hour and fade within a day. Further exposure to UV light causes melanocytes to produce new quantities of melanin from tyrosine, an abundant amino acid in the skin's protein (too much UV light can lead to damage of the proteins that make up the skins connective and elastic tissue leading to sagging and wrinkling). This delayed tan can last for several days without further exposure. Increased exposure to UV sees an increase in the activity and number of melanocytes and the lengthening of the melanin polymer chains.

Hyperplasia

Exposure increases production of skin cells via hyperplasia of the stratum corneum, epidermis, and dermis. UV-induced hyperplasia results from increased epidermal and dermal mitotic activity about 24-48 hours after acute UV exposure and is also associated with increased synthesis of DNA, RNA, and proteins (Epstein, 1970). This temporary thickening of the skin can decrease UV transmission 10 fold.

Sunburn or Erythema

Sunburn or erythema is the most obvious and visible acute cutaneous response to UV irradiation. The molecules responsible for light absorption (chromophores) that initiates sunburn inflammation have not been precisely identified. However, the action spectrum of erythema (290 to 330 nm) is nearly identical to that proposed for DNA damage (Young et al, 1998), suggesting that the principal event would be direct damage to DNA by UVB and short UVA wavelengths (Matsumura and Ananthaswamy, 2004).

DNA Damage

UV irradiation induces DNA lesions such as pyrimidine diners and (6-4) photoproducts, which could lead to DNA mutations and cancer if they are not repaired. To prevent DNA mutations, cells are equipped with a DNA repair mechanism that constantly monitors and repairs most of the damage inflicted by UV light. This system is called the nucleotide excision repair system. In this process, the p53 gene (plays a pivotal role by causing cell cycle arrest to gain some time for DNA repair, or inducing cell death by apoptosis when DNA damage is too severe to repair). Ongoing UV damage to this system and the genes involved can result in an accelerated rate of cellular mutation, potentially leading to genomic instability and cancer.

Conventionally, individuals have supplemented the skin's defence mechanism by the use of sunscreens (as illustrated in Table 1) that absorb/filter or reflect/block incident radiation, primarily UVB and short-length UVA2. However, longer wavelengths of UVA (UVA1) (340-400 nm), have been shown to cause morphological changes is human skin indicative of photo-damage and the induction of skin tumours (Laver & Kaidbey, 1997), and it is considered that the use of the sunscreens described above may result in an increased exposure to long wave UVA1 by selectively changing the spectrum of solar sunlight received by the skin (Autier et al, 1999 and Diffey, 1992).

TABLE 1
Common Sunscreen Ingredients

| PROPERTY | DESCRIPTION |
|---|---|
| UVB FILTERS | UVB filters include para-aminobenzoic acid (PABA) and its |
| | derivatives; cinnamates such as cinoxate, octocrylene and octyl |
| | methoxycinnamate (OMC), bezophenones and salicylates. These |
| | work by absorbing UVB light but none of these offer significant |
| | protection against UVA radiation. Dibenzoylmethane derivatives |
| | and anthranilates are the exception by offering UVB absorbtion |
| | and mild UVA aborbtion. |
| UVA FILTERS | UVA filters, octocrylene and benzophenones, a family of |
| | chemicals including oxybenzone, dioxybenzone and |
| | butylmethoxydibenzoylmethane, are commonly used in |
| | suncreams to absorb UVA light but work at shorter UVA |
| | wavelengths. Avobenzone (Parsol 1789 ©) that works against all |
| | UVA and UVB wavelengths. |
| SUNBLOCKS | Zinc and titanium oxide are mineral-derived sun blocks which |
| | reflect light, bouncing it away from the skin. These offer |
| | significant UVA and UVB protection. |
| | Mineral sunscreens were traditionally very messy and tended to |
| | leave a visible white film, but the new high-tech formulations |
| | contain fine micro-pigments (e.g BASF Z-COTE ® transparent |
| | zinc oxide) which make them smoother, light and easy to blend. |
| SUNBURN | Many suncreams contain salicylates - aspirin-like chemicals |
| PREVENTERS | which help prevent sunburn. Commonly used salicylates are |
| | ethylhexyl salicylate, homosalate, octyl salicylate, isotridecyl |
| | salicylate and neohomosalate. |
| SKIN | Skin protectors such as PABAS, including p-aminobenzoic acid, |
| PROTECTORS | ethyl dihydropropyl PABA, padimate-O, padimate A and |
| | glyceryl PABA, are used in suncreams to help prevent skin |
| | damage. |
| | They also have self-plasticising properties that form a continuous |
| | plastic layer on the skin. NB amino benzoates not as optically |
| | efficient as benzophenones but don't crystallise as easily so form |
| | better film and adhere to the skin. |
| PRESERVATIVES | Parabens are among the most widely used preservatives. |
| | Trisodium Edta is another preservative used in suncreams to |
| | prevent titanium or zinc oxide from breaking down and not |
| | working properly. |

An alternative approach for supplemental protection from the harmful effects of the suns rays, is the use of fabrics that provide UV protection. Such fabrics can be manufactured into articles of clothing and also non-apparel articles such as tents, awnings, crowd covers and parasols. U.S. Pat. No. 5,414,913 and U.S. Pat. No. 5,503,917 for example disclose fabrics which reduce the transmission of UVA and UVB by the alteration of the ratio of threads to apertures. The incorporation of dyes for increasing the sun protection factor (SPF) rating of a fabric is disclosed in WO 9625549, WO 9417135 and WO 9404515. WO 02059407 discloses a fabric comprising synthetic polymers in which the fabric has been calendered or “chintzed” on at least one surface in order to improve the UV protection factors. A further method is the incorporation of UV blocking particles or absorbers into fabrics, these particles reflect, absorb and/or scatter the UV rays, such an approach is outlined in U.S. Pat. No. 6,037,280 and EP 0 919 660.

Whilst the fabrics disclosed in the prior art can be used to manufacture articles of clothing for an individual to wear and thereby limit the amount of skin exposure to UV light, it is not possible to completely cover up from exposure to the sun, particularly in countries with warm climates. If an individual has moles on exposed sites of skin such as on the face, scalp and hands it would be clinically valuable to be able to limit the amount of UV that these sites are exposed to.

There is a need for a means to focally protect vulnerable areas of the skin (e.g moles) against the harmful effects of exposure to UV radiation, and thereby prevent or diminish the likelihood of the development of cancer in these areas. The application of a patch gives the user the reassurance that specific areas of the skin are protected from UV until the patch is removed and they therefore do not, for example, have to worry about the re-application of sunscreens.

### STATEMENT OF THE INVENTION

Thus, according to an aspect of the invention there is provided a patch comprising a first layer which is adhesive and a second layer comprising a material adjacent to said first layer characterised in that at least one of said first or second layers is opaque to UV radiation

The term opaque as herein used is defined as being substantially impenetrable by a form of radiation other than visible light. The term UV radiation as herein used is defined as wavelengths in the ultra violet spectrum, UVA (320-400 nm), UVB (280-320 nm) and UVC (200-280 nm).

Preferably the patch is substantially opaque to UVA, UVB and UVC radiation thereby significantly reducing w transmission. In an alternative embodiment of the invention the patch is opaque to UVA and UVB radiation. UVA and UVB radiation have been found to be the elements of the UV spectrum that cause deleterious medical effects. Whilst the UVC component has the potential to induce deleterious medical effects it is largely removed by the ozone layer. However, as the ozone layer is depleted, particularly over areas such as Australia, the need to protect against UVC radiation will increase significantly.

The first layer of the patch, which is the adhesive layer may be opaque to UV radiation, but it is preferably the second layer of the patch that is opaque to UV radiation.

A UV protection factor (UPF) below 15 is deemed “low protection”, a UPF of 15 to 30 is deemed “medium protection” and a UPF greater than 40 is deemed “high protection”.

Preferably the patch has a UPF of equal to or greater than 40. Alternatively said patch has a UPF in the range of from 15-40.

The protection against UV light can also be described in terms of sun protective factor (SPF). An SPF number is measured by the following equation: 100/% transmission of UV light=SPF number. Thus a composition permitting 20% transmission has a SPF # of 5; whilst a composition permitting 10% transmission has a SPF # of 10.

The opaque property of the first and/or second layer of the patch is preferably as a result of a chemical or physical modification.

Even more preferably said chemical modification comprises UV blocking agents. Examples of UV blocking agents are described in U.S. Pat. No. 6,037,280 which is herein incorporated by reference. UV blocking agents act as a result of absorbing, filtering, deflecting, reflecting, absorbing or scattering the UV radiation.

The UV radiation blocking agents are preferably inorganic, organic or metallic. Examples of such agents include, but are not limited to; muscovite, phlogopite, biotite, sericite, fushitite, margarite, synthetic mica, metal oxide coated mica, coloured pigment coated mica, talc, benzotriazole e.g chlorobenzotriazoles, para-aminobenzoic acid, metal oxides, metallic hydroxides, mixed metal oxides and hydroxides, metal and mixed metal silicates and aluminosilicates, transition metal oxides and hydroxides, Ti02, Zr02, Fe2O3, natural clay, metal sulfides, non-metallic elements, ionic salts and covalent salts, powered ceramics, organic polymers for example CYASORB® (Cytec Technology Corp, USA) UV-3346, -1164, -3638, -5411 and TINUVIN® (Ciba Specialty Chemicals Holding Inc., Switzerland), natural polymers, insoluble organic materials and biomaterials, particularly UV absorbing molecules, aluminium, copper, copper-bronze, bronze-gold, silver and collagen.

In a preferred embodiment of the invention the metallic agent is a zinc salt. Even more preferably the zinc salt is zinc sulphide or zinc oxide.

Preferably said UV radiation blocking agent absorbs UV radiation and is para-aminobenzoic acid (PABA). This compound is a UV absorber found in tanning lotions.

Preferably the UV radiation blocking agent is a particle. A binding agent may be used to bind the UV radiation blocking particle to the fabric. Examples of such binding agents include but are not limited to casein isolate, soy protein isolate, starch, starch derivatives, gums and synthetic latexes.

Preferably the UV radiation blocking agents are incorporated within a layer of the patch. Even more preferably still said incorporation is within interstitial spaces. Alternatively the UV radiation blocking agents are attached to a surface of a layer. Preferably this surface is an upper surface of the second layer.

The above described agents may be applied to a layer using techniques known to those skilled in the art, for example via conventional rotogravure or flexographic coating processes using solvent or water based carrier systems. The carrier systems may also be UV curable. The agents may be in tablet form, delivered from a sachet, bottle, tube or other mechanisms for delivering such agents in a concentrated form, such as a paste.

In an alternative embodiment of the invention at least one of said first or second layers of said patch is opaque to UV radiation as a result of a physical change within said layer.

The permeability of a fabric is an important factor in opaqueness to UV radiation. Conventionally a fabric can be made relatively UV-opaque by providing a relatively tight weave or a very high thread count, or by coating the fabric.

Calendering or chintzing is a known technique for improving the wind resistance of certain materials, for reducing the leakage of fibres through a fabric from a fibrous insulation layer or for changing the appearance of certain fabrics. Calendering or chintzing is performed by applying heat and pressure to at least one surface of a fabric. Calendered surfaces are easily identified by the characteristic plastic deformation of the surface. The calendaring temperature is preferably maintained in a range of 140° C. to 195° C. The calendering pressure is preferably 50 tonnes/sq.inch (6.5×10⁶ N/m²) (±(10%). And the calendaring is preferably performed at a speed in the range of from 12 to 18 meters per minute.

Therefore, preferably said structural change is achieved by calendering as described in PCT/GB02/00317, as herein incorporated by reference. Even more preferably said second layer is calendered on at least one surface.

Alternative methods of inducing such a structural change to enhance the UV protection factors include but are not limited to sanding followed by jet laundering as disclosed in U.S. Pat. No. 5,503,917 or ‘wrinkling’ of the fabric as disclosed in EP 0919 660.

Specific weaves, twists or bends of yams or fabrics which have been developed to effectively screen UV radiation is disclosed in U.S. Pat. No. 4,861,651.

Moles on the skin have been shown to be particularly prone to developing into a melanoma. It is therefore particularly advantageous to be able to apply a patch according to the invention directly above such a mole. However, it is desirable that the adhesive itself is not brought into direct contact with the mole as this may be potentially harmful to the mole, particularly when the patch is removed. Preferably therefore the adhesive is provided at a peripheral edge of the patch and the extremity of the patch extends beyond the extremity of the mole. Even more preferably still, the patch is substantially circular and the adhesive is provided around the peripheral circumference of the patch.

The patch may be manufactured of sufficient size to extend over a plurality of moles, for example, if a discrete group of moles exist on the skin.

Even more preferably the adhesive is provided with a releasable protective layer, which is removed only when the patch is to be applied to the skin.

Preferably the second layer of the patch substantially overlies the first layer. Even more preferably the first layer comprises a substantially single thickness fabric. The term ‘single thickness fabric’ is defined as a single woven, non-woven or knitted layer of textile filaments. Even more preferably still said second layer comprises a section of tape or film. The patch may be manufactured as a single piece of tape to which an adhesive is applied.

It may be desirable to the user to apply the patch to visible areas of the skin, such as the head, neck and scalp. In order to render the patches as unobtrusive as possible on the user's skin it is preferable that the patch is transparent to visible light. The use of a transparent material which is impermeable to UV penetration has been used in the field of contact lenses, contact lenses have been developed with incorporate UV blockers and are designed to complement sunglass use as an added protection.

In an alternative embodiment of the invention the second layer of the patch comprises a gel. Preferably the gel rests above the mole when the patch is applied. In this embodiment the first layer may comprises a fabric such as a piece of tape which is provided with an adhesive. The gel comprises UV blocking agents as described in the first embodiment of the invention.

In a further aspect of the invention there is provided the use of a patch according to the invention as a preventive agent against the development of melanoma

In a further aspect of the invention there is provided a method of manufacturing a patch wherein the patch comprises a first layer which is adhesive and a second layer adjacent to the first layer characterised in that at least one of the first or second layers is opaque to UV radiation; comprising the steps of;
i) providing a first and second layer wherein at least one of the layers is opaque to UV radiation or capable or being rendered opaque to UV radiation;
ii) bringing into contact the layers in (i).

Preferably the second layer comprises a single thickness fabric. Even more preferably still the single thickness fabric is a section of tape or film. Alternatively the second layer comprises a gel.

Even more preferably a releasable protective layer is applied to the adhesive to prevent the patch sticking to other materials prior to application to the skin.

Even more preferably still the patch may also be inserted into a wrapper for storage prior to use.

The opaqueness is a result of a modification of at least one layer of the patch, said modification being selected from the group consisting of chemical or physical modification.

Examples, which are not limiting of chemical and physical modifications are outlined above. Preferably said a chemical modification comprises the addition of at least one UV reflecting and/or absorbing agent to at least one layer of the patch. Preferably the physical modification is as a result of calendaring of at least one layer of the patch.

In a further aspect of the invention there is provided a method of reducing skin exposure to UV radiation comprising the steps of;
i) providing a patch comprising a first layer which is adhesive and a second layer adjacent to said first layer characterised in that at least one of said first or second layers is opaque to UV radiation;
ii) applying said patch to the skin with the adhesive layer contacting said skin.

In a further aspect of the invention there is provided a method of preventing skin cancer as a result of exposure to UV radiation comprising the steps if;
i) providing a patch comprising a first layer which is adhesive and a second layer adjacent to said first layer characterised in that at least one of said first or second layers is opaque to UV radiation;
ii) applying said patch to an area of skin with the adhesive layer contacting said skin.

Preferably the skin cancer is selected from the group consisting of basal cell carcinoma, squamous cell carcinoma or malignant melanoma.

Preferably the area of skin is specifically susceptible to UV radiation. Even more preferably still the area of skin is a mole.

According to a still further aspect of the invention there is provided a kit comprising a plurality of patches of varying shapes and sizes.

An embodiment of the invention will now be described by example only and by reference to the accompanying Figures, Materials and Methods.

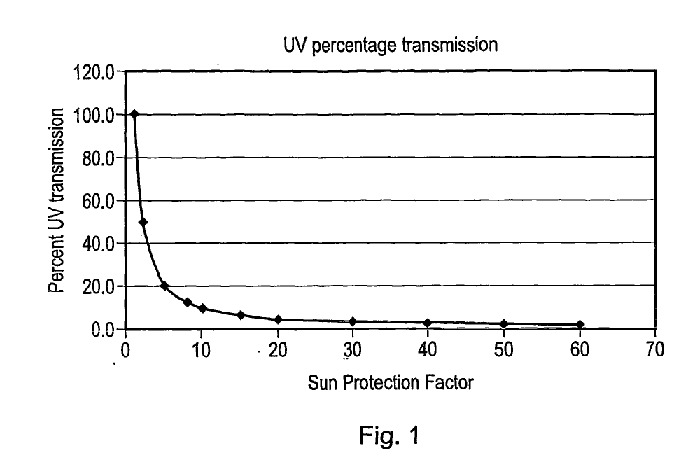

FIG. 1: Illustrates the relationship between Sun Protection Factor (SPF) and Percentage UV transmission.

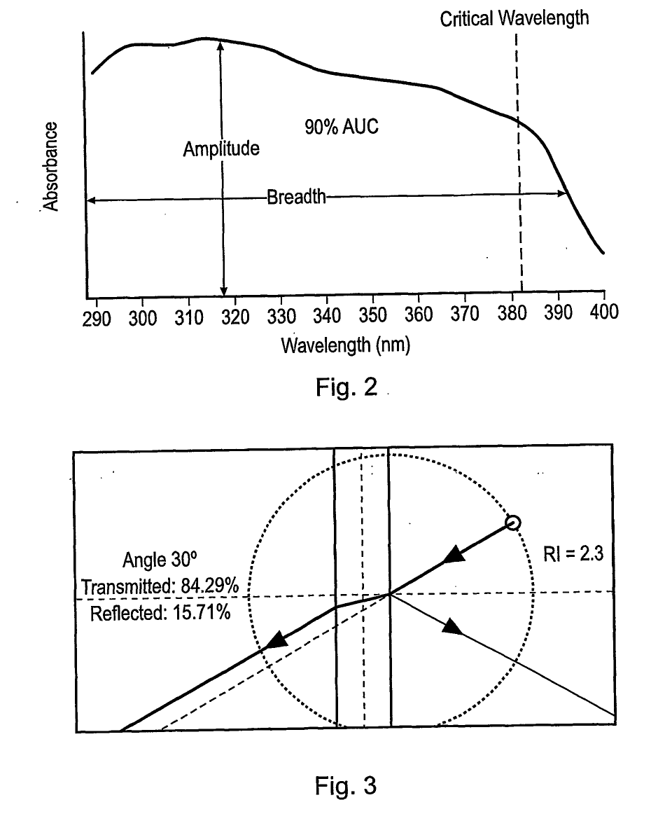

FIG. 2: Illustrates the absorption spectrum for a hypothetical sunscreen product. UV attenuation is determined at fixed intervals across UV spectrum using substrate spectrophotometry. Wavelength below which 90% of the area under the whole absorption spectrum from 290 to 400 nm falls in the critical wavelength. The shape if the absorption spectrum is independent of application density.

FIG. 3: Illustrates the affect of the refractive index on reflection and transmission. When light is coming in perpendicular to a film surface, very little of it is reflected. This reflection grows in proportion as the angle of incidence increases; at first slowly and then dramatically, until a point where all incident light is reflected. This angle is identified as the cut off angle.

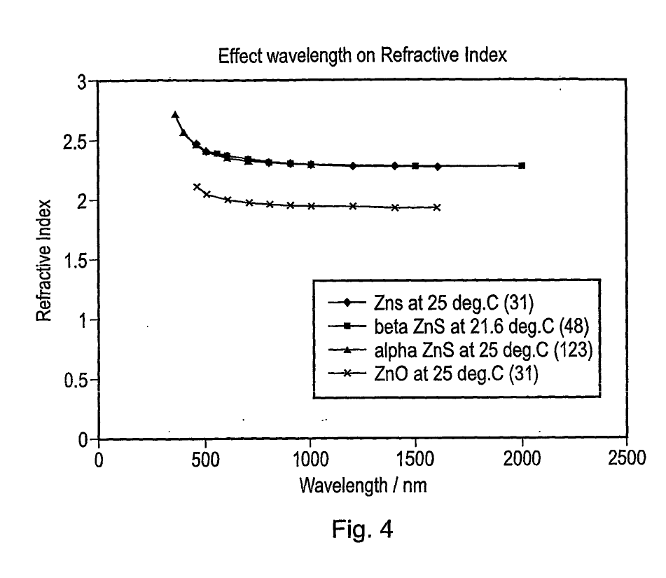

FIG. 4: Illustrates the effect of wavelength on the refractive index.

### MATERIALS AND METHODS

Measurement of UV Protection

Industry standards for characterising the degree of protection from UV radiation have largely been defined in association with the sunscreen industry. This has led to a myriad of test methods and ratings as outlined below:

Percentage Transmission

The amount of light a material will let through (at a given wavelength) depends on the thickness of the material (d cm), the concentration of the material (c g/L), and the absorption coefficient (a) The relationship given between the light falling on the surface of a material (Iₒ) and the amount of light transmitted (I) is given by Beers Law:

$$Log \frac{I}{{I}_{o}} = - a \cdot c \cdot d$$

I/Iₒ is called the transmittance. It can be expressed as a percentage, 100 I/Iₒ called the per cent transmittance.

$$\% T = 100 \frac{I}{{I}_{o}}$$

Sun Protection Factor (SPF)

Provides an index of protection against erythemally effective solar UV largely confined to UVB (290-320 nm) and short wavelength UVA (320-340 nm). this is given as the Sun Protection Factor (SPF), see Table 2 and FIG. 1.

After about 20 minutes exposure to the midday sun, an average untanned white skin will be affected by sunburn although the actual reddening will not appear until after about 6 hours. The reddening will still be able to be seen 24 hours later. The exposure needed to give this effect is known as the minimum erythemal dose (MED). By comparing the time necessary to produce this MED on unprotected skin to that needed to produce it on skin protected with a standard amount of sunscreen it is possible to give a sun protection factor (SPF) for the sunscreen (independent of the absolute intensity of the radiation).

$$PF = \frac{exposure duration for MED in protected skin}{exposure duration for MED in unprotected skin}$$

A factor of 10 means a person can stay out in the sun about 10 times longer than without a sunscreen and achieve the same effect.

The protection factor should be proportional to the quantity of UV light transmitted through the layer of sunscreen to the skin. So, if the sunscreen has a transmittance (T) of 50% it should provide SPF 2 and when transmittance is 10% it should provide SPF 10.

TABLE 2
SPF Factor

| SPF | % UVB Transmission |
|---|---|
| 1 | 100.0 |
| 2 | 50.0 |
| 5 | 20.0 |
| 8 | 12.5 |
| 10 | 10.0 |
| 15 | 6.7 |
| 20 | 5.0 |
| 30 | 3.3 |
| 40 | 2.5 |
| 50 | 2.0 |
| 60 | 1.7 |

Sun Protection Factor Test

The test method recommends that at least 10 healthy, fair skinned volunteers are used in the testing. The volunteers must be pre-screened to find out their MED. The same amount of sunscreen should be applied to each volunteer so that the results are reliable. 1 mg/cm2 of sunscreen should be applied to the skin. The test is restricted to the back. Each person is then exposed to controlled amounts of simulated sunlight.

The SPF of a product is calculated as the arithmetical mean of the individual sun protection factors.

Determination of Transmission from 320 nm to 360 nm—Broad Spectrum Products

Method 1—Solution Method

Used for products that dissolves completely in a spectroscopic grade solvent of dichloromethane, Cyclohexane and Isopropanol.

Transmission of an 0.8 mg/mL organic solvent solution of the sample in a 10 mm thick cell is measured between 320 nm and 360 nm by a spectrophotometer. The sample should not allow more than 10% transmission at any point. (Reported as Pass or Fail ).

Method 2—Thin Film Method

Used for products that are opaque by reflection rather than absorption or which does not completely dissolve in solvent used in Test Method 1.

Transmission of an 8 μm film is measured between 320 nm and 360 nm by a spectrophotometer. The film should not allow more than 10% transmission at any point over the range (Reported as Pass or Fail).

Method 3—Plate Method

Used for all products regardless of the solvents used and whether the sunscreen includes suspended solids.

Transmission of 20 μm plastic film is measured between 320 and 360 nm by a integrated sphere spectrophotometer or a near ultraviolet radiometer. The film should not allow more than 1% transmission at any point over the range (Reported as Pass or Fail).

Star Rating Test Method

Provides an index of protection against UVB (290-320 nm) and both short and long-wavelength UVA (320-400 nm).

Absorption of a 2 mg/sq cm film is measured between 290 nm and 400 nm. Pre irradiation of the sample IS NOT required. The rating scale is 1 to 4 stars. One Star means the product only offers a quarter of the protection against UVA as it offers against UVB. Four Stars means it offers the same.

UVA-Protection Factor (PF)

UVA-PF is a proposed new method that aims to standardize the Star Rating Test Method outlined above and so is subject to change. Absorption of a 0.75 mg/sq cm film is measured between 290 nm and 400 nm (both UVB and UVA). The ratio of areas under the curve between 290-320 (UVB region) is compared with the area under the curve between 320 and 400 nm. Adjustment is made for products with SPF above 30, so that they are not disadvantaged. i.e. for above SPF 30, the ratio only needs to be 12. Pre irradiation of the sample is required. (Calculated as SPF×UVA/UVB).

Critical Wavelength

Critical Wavelength is the current proposed in vitro method for measuring protection against the whole UVB and UVA wavelengths (290-400 nm). Absorption of an 0.75 mg/sq cm film is measured between 290 nm and 400 nm. The critical wavelength is the point where 90% of the area under the spectral absorbance curve lies, starting at the UVB end. Pre irradiation of the sample is required. (Reported as SOME (UVA/UVB)—between 340 nm and 370 nm, MORE (Broad Spectrum)—above 370 nm.

The critical wavelength value is based on the inherent shape of the absorbance curve not its amplitude and therefore is independent of application thickness (see FIG. 2). Diffey et al, 2000 assessed the critical wavelengths of 59 sunscreens using the following materials and methods. A hydrated synthetic collagen substrate is used to simulate human skin. 1 mg/cm2 of product is applied to the hydrated synthetic collagen. Samples are pre-irradiated with broad-band UV radiation (290-400 nm) using an xenon arc solar simulator and filters and the UV absorbance of the product film was measured using a UV substrate spectrophotometry (e.g Labsphere UV-1000S transmittance analyser).

Multiple determinations from 5 independent samples per product were used to calculate the critical wavelength value, defined as the wavelength at which the integral of the spectral absorbance curve reached 90% of the integral from 290 to 400 nm. The final critical wavelength value for each product was the 95% lower confidence limit computed from the 5 individual replicates.

International and National Testing Standards

There are a number of standard tests for determining UV absorbance which would be readily accessible to the skilled person. Examples are;

BS EN 1836:1997 Personal eye protection. Sunglasses, sunglare filters for general use and filters for direct observation of the sun.

BS EN 13758-1:2002 Textiles—Solar UV protective properties—Part 1: Method of test for apparel fabrics

BS EN 13758-1:2002 Textiles—Solar UV protective properties—Part 2: Classification and marking of apparel

AS/NZS 4399:1996: Sun Protective clothing—Evaluation and classification. The UVR transmitted through a specimen is collected in an integrating sphere and determined in the range 290 nm to 400 nm. UPF is determined and the mean UVA and UVB transmittance is also reported.

AS/NZS 2604:1998 Sunscreen products—Evaluation and classification.

Solar simulator with properties that can be achieved by the use of the xenon arc with filters is used in this Standard.

AS/NZS 4174:1994 Synthetic Shadecloth. The UVR transmitted through a specimen is collected in an integrating sphere and determined in the range 290 nm to 770 nm. Mean Ultraviolet Radiation (UVR 290 to 400 nm), Photosyntetically active radiation (PAR 400 to 700 nm) and UVR block are reported.

AATCC Test Method 183-2000 Transmittance or Blocking of Erythemally Weighted Ultraviolet Radiation through Fabrics

ASTM E903-96 Standard Test Method for Solar Absorptance, Reflectance, and Transmittance of Materials Using Integrating Spheres

ASTM E424 Test Methods for Solar Energy Transmittance and Reflectance (Terrestrial) of Sheet Materials

ASTM E1084 Test Method for Solar Transmittance (Terrestrial) of Sheet Materials using Sunlight

The construction of the Patch

Mole patch construction requires a film substrate component to block/reflect UV transmission. Taking the clothing industry norm, a sun protection factor of 40 i.e. a 2.5% transmission of UV frequencies through these materials would be desirable.

The patch comprises a refraction/reflection component and/or an absorption component;

The UV Refractive/Reflective Component

Transparent, high refractive index coated films offer the potential to reduce UV transmission through films. These film types are commercially available (usually using zinc sulphide or titanium oxide coatings).

The Effect of Refractive Index on Reflection

Refraction occurs between transparent materials of different densities, such as air and glass. The bending evident in refraction is a physical representation of the longer time it takes light to move through the denser of the two materials, and it is dependent on the angle that the light strikes the boundary.

Refraction is dependent on two factors: the incident angle (q) and the refractive index (n) of the material, as given by Snell's law of refraction.
n sin(q)=n′ sin(q′)

The refractive index is a constant for a given transparent material. Different wavelengths are refracted different amounts in a given material so it usually has different values for different wavelengths of light.

Compared to the overall electromagnetic spectrum, visible light frequencies make up only a very small band so it is usually given as a single value. However, this small difference can cause the UV end of the spectrum to refract more than the infra red through certain materials (see FIG. 3).

The impact of refractive index of material on proportion of reflected light (data for visible light frequencies) is shown in Table 3 below;

TABLE 3

| Incident angle | RI = 1.5 | RI = 2.0 | RI = 2.5 | RI = 3.0 |
|---|---|---|---|---|
| 1 | 4.03 | 11.18 | 18.32 | 24.99 |
| 10 | 4.03 | 11.18 | 18.32 | 24.99 |
| 30 | 4.18 | 11.35 | 18.45 | 25.07 |
| 50 | 5.81 | 13.15 | 19.71 | 25.77 |
| 70 | 16.78 | 23.33 | 27.47 | 30.81 |

The patch can consist of at least one common polymer selected from the group represented in Table 4 below. This Table illustrates the Refractive index data for these common polymers (visible spectrum data) taken from the literature http://www.plasticsusa.com/refract.html, http:/www.texloc.com/closet/cl_refractiveindex.html).

TABLE 4
Common polymers

| Material | Refractive Index |
|---|---|
| Fluorcarbon (FEP) | 1.34 |
| Polytetrafluoro-Ethylene (TFE) | 1.35 |
| Cellulose Propionate | 1.46 |
| Cellulose Acetate Butyrate | 1.46-1.49 |
| Cellulose Acetate | 1.46-1.50 |
| Methylpentene Polymer | 1.485 |
| Ethyl Cellulose | 1.47 |
| Acetal Homopolymer | 1.48 |
| Acrylics | 1.49 |
| Cellulose Nitrate | 1.49-1.51 |
| Polypropylene (Unmodifiedd) | 1.49 |
| Polyallomer | 1.492 |
| Polybutylene | 1.50 |
| Ionomers | 1.51 |
| Polyethylene (Low Density) | 1.51 |
| Nylons (PA) Type II | 1.52 |
| Acrylics Multipolymer | 1.52 |
| Polyethylene (Medium Density) | 1.52. |
| Styrene Butadiene Thermoplastic | 1.52-1.55 |
| PVC (Rigid) | 1.52.-1.55 |
| Nylons (Polyamide) Type 6/6 | 1.53 |
| Urea Formaldehyde | 1.54-1.58 |
| Polyethylene (High Density) | 1.54- |
| Styrene Acrylonitrile Copolymer | 1.56-1.57 |
| Polyethylene terephthalate | 1.57 |
| Polystyrene (Heat & Chemical) | 1.57-1.60 |
| Polycarbonate (Unfilled) | 1.586 |
| Polystyrene (General Purpose) | 1.59 |
| Polysulfone | 1.633 |
| Polyetheretherketone (amorphous) | 1.65-1.71 |
| Zinc Sulphide (Lu, 2003) | 2.36 |
| Titanium Dioxide (Huang, 2004) | 2.3-2.4 |

Base film substrates typically have refractive indices in the range 1.4-1.6. The reflective component associated with light refraction through these films will only reduce light transmission through these materials by an order of approximately 5% (i.e. 95% transmission).

The application of inorganic coatings can yield much higher refractive indices. Zinc sulphide and titanium oxide yield refractive indices in the range 2.3-2.4 for visible light frequencies. Again referring to reference tables, light transmission through these materials will be reduced by approximately 18% (82% transmission).

The refractive index of a material generally increases at lower wavelengths. Tables 4 and 5b and also FIG. 4 summarise the impact of incident light frequency on refractive index.

TABLE 4

| | |
|---|---|
| TiO2 | Wavelength |

| 25deg. C. | nm | RI |
|---|---|---|
| | 436 | 2.853 |
| | 546 | 2.652 |
| | 691 | 2.555 |
| | 708 | 2.548 |
| | 1014 | 2.484 |
| | 1530 | 2.454 |

TABLE 5

| | nm | ZnS | ZnO |
|---|---|---|---|
| | 450 | 2.47 | 2.11 |
| 25deg. C. | 500 | 2.42 | 2.05 |
| | 600 | 2.36 | 2 |
| | 700 | 2.33 | 1.97 |
| | 800 | 2.31 | 1.96 |
| | 900 | 2.3 | 1.95 |
| | 1000 | 2.29 | 1.94 |
| | 1200 | 2.28 | 1.94 |
| | 1400 | 2.28 | 1.93 |
| | 1600 | 2.27 | 1.93 |

Refractive Index data for ZnS in the UV region suggests an increase to around 2.8-2.9⁴. This will lead to a reduction of light transmission at these frequencies of up to 25% by reflection (i.e. 75% transmission).

The UV Absorptive Component

UV transmission data for common base films are given in Table 6.

TABLE 6

| | 320-390 nm | 280-320 nm | 250-260 nm |
|---|---|---|---|
| | UVA | UVB | UVC |
| Polypropylene | 85 | 79 | 71 |
| PET | 88 | 29 | 66 |
| PVC | 85 | 71 | 64 |

PET is far more effective than PVC and polypropylene at screening UVB frequencies. This is associated with the aromatic ring structures present within these materials.

Polycarbonate polymers have more extensive aromatic group functionalities. These polymers are commonly used in safety spectacles where UV protection is required. Technical data sheets for GE Materials “Lexan” polycarbonate sheet product infer a UV transmission of 0%. Polycarbonate is normally produced in rigid sheet form, and one source is Piedmont Plastics.

There are a number of materials known to the man skilled in the art that can be used for this purpose. For example contact lenses use coatings of benzotriazole. Manufacturers data quote 98% UV absorption for these products. Recognised UVA1 (340-400 nm) filters include avobenzone, zinc oxide, or titanium dioxide. Other UV absorbing filters that have been quoted in the literature include: muscovite, phlogophite, biotite, sericite, fushitite, margaite, synthetic mica, metal oxide coated mica, coloured pigment coated mica, talc, benzotriazole (e.g chlorobenzotriazoles), benzoates and benzophenone, para-aminobenzioc acid (ABA), TiO₂, ZrO₂, Fe₂O₃, natural clay, organic polymers (e.g CYASORB™ by Cytec, TINUVIN™ and CHIMASSORB™ by Ciba, EVERSORB™ by Everlight USA, LOWILITE™ by Great Lakes).

FIG. 6 shows the absorption bands and critical wavelength for the most commonly used UV filters.

A typical patch may consist of a simple base film (e.g PET polyester) or coated films, for example Llumar™ films (a division of CP Films), which are widely used in the architectural and automotive industries.

Table 7 illustrates the UV transmission properties associated with a number of Llumar™ films.

TABLE 7

| | | % Solar | | % Solar | % UV |
|---|---|---|---|---|---|
| | | Trans- | % Solar | absorp- | trans- |
| LLumar | Colour | mission | reflection | tion | mission |
| SCL ER | Clear | 84 | 9 | 7 | Max 5 |
| PS4 | | | | | |
| REX50 SI | silver | 37 | 33 | 30 | Max 1 |
| ER HPR | | | | | |
| NEX20 | Stainless | 22 | 29 | 49 | Max 1 |
| SSER HPR | steel | | | | |
| NEX1020 | Copper | 12 | 71 | 17 | Max 1 |
| SB ER | bronze | | | | |
| HPR | | | | | |
| V14 ER | neutral | 8 | 59 | 33 | Max 1 |
| HPR | | | | | |

A further approach involves utilising the UV absorbing properties of some of the high refractive index inorganic coatings.

1. A patch comprising a first layer which is adhesive and a second layer comprising a material adjacent to the first layer characterised in that at least one of the first or second layers is opaque to a UV radiation.

2. A patch according to claim 1, wherein the second layer is opaque to a UV radiation.

3. A patch according to claims 1 or 2, wherein the UV radiation is selected from the group consisting of (320-400 nm), UVB (280-320 nm) and UVC (200-280 nm) radiation.

4. A patch according to any of claims 1 to 3, wherein the patch has a UV protection factor (UPF) greater or equal to 40.

5. A patch according to any of claims 1 to 3, wherein the patch has a UV protection factor (UPF) in the range of from 15 to 40.

6. A patch according to any of claims 1 to 5, wherein at least one layer comprises a modification which results in the layer being opaque to UV radiation, wherein the modification is chemical or physical.

7. A patch according to claim 6, wherein the chemical modification comprises the addition of UV radiation blocking agents.

8. A patch according to claim 7, wherein the second layer comprises the UV radiation blocking agents.

9. A patch according to claims 7 or 8, wherein the UV radiation blocking agents is incorporated into a layer.

10. A patch according to claim 9, wherein the incorporation is with interstitial spaces within a layer.

11. A patch according to claims 7 or 8, wherein the UV radiation blocking agents is attached to a surface of a layer

12. A patch according to any of claims 7 to 11, wherein the UV radiation blocking agents act as a result of deflecting and/or reflecting and/or absorbing and/or scattering the UV radiation.

13. A patch according to any of claims 7-12, wherein the UV radiation blocking agents are selected from the group consisting of; inorganic, organic or metallic agents.

14. A patch according claim 13, wherein the metallic agent is a zinc salt.

15. A patch according to claim 14, wherein the zinc salt is zinc sulphide or zinc oxide.

16. A patch according to claim 7, wherein the physical modification is a result of calendaring.

17. A patch according any of claims 1 to 16, wherein the adhesive is provided at a peripheral edge of the patch.

18. A patch according to claim 17, wherein the adhesive is provided with a releasable protective layer.

19. A patch according to any of claims 1 to 18, wherein the second layer substantially overlies said first layer.

20. A patch according to any of claims 1 to 19, wherein the material comprises a substantially single thickness fabric.

21. A patch according to claim 20, wherein the material comprises a section of tape or film.

22. A patch according to any of claims 1 to 19, wherein the material comprises a gel.

23. A patch according to any of claims 1 to 22, wherein said patch is substantially circular.

24. A patch according to any of claims 1 to 23, wherein said patch is substantially waterproof.

25. A patch according to any of claims 1 to 24, wherein said patch is substantially transparent to visible light.

26. The use of a patch according to any of claims 1 to 25 as a preventive agent against the development of melanoma.

27. A method of manufacturing a patch wherein said patch comprises a first layer which is adhesive and a second layer adjacent to the first layer characterised in that at least one of the first or second layers is opaque to UV radiation comprising the steps of;
i) providing a first and second layer wherein at least one of the layers is opaque to UV radiation or capable or being rendered opaque to UV radiation;
ii) bringing into contact the layers in (i).

28. A method according to claim 27, wherein the second layer is a single thickness fabric.

29. A method according to claim 28, wherein the single thickness fabric is a section of tape or film.

30. A method according to claim 27, wherein the second layer is a gel.

31. A method according to any of claims 27 to 30, wherein a releasable protective layer is applied to the adhesive.

32. A method according to any of claims 27 to 31, wherein the opaqueness is a result of a modification of at least one layer, the modification being either a chemical or physical modification.

33. A method according to claim 32, wherein the chemical modification comprises UV radiation blocking agents.

34. A method according to claim 32, wherein the physical modification is as a result of calandering.

35. A method of reducing skin exposure to UV radiation comprising the steps of;
i) providing a patch comprising a first layer which is adhesive and a second layer adjacent to the first layer wherein at least one of said first or second layers is opaque to UV radiation;
ii) applying the patch to the skin with the adhesive layer contacting the skin.

36. A method of preventing skin cancer as a result of exposure to UV radiation comprising the steps if;
i) providing a patch comprising a first layer which is adhesive and a second layer adjacent to the first layer wherein at least one of the first or second layers is opaque to UV radiation;
ii) applying the patch to the skin with the adhesive layer contacting the skin.

37. A method of preventing skin cancer according to claim 36, wherein the skin cancer is selected from the group consisting of basal cell carcinoma, squamous cell carcinoma, malignant melanoma.

38. A method according to claim 35 or 36, wherein said patch is applied directly to at least one mole.

39. A kit comprising a plurality of patches of varying shapes and/or sizes according to any of claims 1 to 25.
